(19) United States
(12) Patent Application Publication
(10) Pub. No.: US 2026/0090181 A1
(43) Pub. Date: Mar. 26, 2026

(54) PHOTODETECTOR DEVICE WITH AN OPTICAL GUIDE ELEMENT

(71) Applicant: Commissariat à l'Énergie Atomique et aux Énergies Alternatives, Paris (FR)

(72) Inventors: Benoit Racine, Grenoble (FR); Guillaume Blanquer, Grenoble (FR); Anne Planat-Chretien, Grenoble (FR); Christelle Laugier, Grenoble (FR)

(73) Assignee: Commissariat à l'Énergie Atomique et aux Énergies Alternatives, Paris (FR)

(21) Appl. No.: 19/333,555

(22) Filed: Sep. 19, 2025

(30) Foreign Application Priority Data
Sep. 24, 2024 (FR) FR2410191

(51) Int. Cl. H10K 30/87; A61B 5/00

(52) U.S. Cl. CPC H10K 30/87; A61B 5/0059

(57) ABSTRACT

A photodetector device including: an optical guide element; an electronic component for converting light into an electrical signal, configured to detect light on the side of a first surface of the electronic component arranged in front of the optical guide element and on the side of a second surface of the electronic component opposite to the first surface; a reflective layer arranged on the side of the second surface of the electronic component; an optical component arranged between the reflective layer and the optical guide element and at least partially transparent to the light to be detected by the electronic component; and wherein the reflective layer forms a reflective surface conformal to a non-planar surface of the optical component having the reflective layer arranged thereon.

### CROSS REFERENCE TO RELATED APPLICATIONS

This application claims priority to French application number FR2410191, filed Sep. 24, 2024. The contents of this application is incorporated by reference in its entirety.

### TECHNICAL FIELD

The present disclosure generally concerns photodetector devices, or photoreceptors, with optical guide elements, or light guide elements.

### PRIOR ART

Light is a measurement modality increasingly used in the healthcare field to determine, or diagnose, and track, or monitor, physiological parameters of interest, such as people's heart rate, blood oxygen or blood sugar levels. To achieve this, light is injected through the epidermis and then collected after having interacted through absorption and/or scattering processes with the tissue of interest, enabling to measure the targeted physiological parameters. The collected light is however very low, due to the high rate of absorption and scattering of light in tissue.

It is known to use organic photodiodes (OPDs) to receive and capture light, and transform it into a measurable quantity corresponding to an electrical signal. OPDs have now reached performance levels which enable them to be used in different products and fields of application. The use of organic materials to perform photodetection provides a number of advantages over semiconductor photodiodes: access to all the intrinsic properties of these materials, to the geometry of the OPD and to the absorption wavelengths, and possibility of deposition on flexible substrates allowing, for example, an optimal adjustment of the OPD to a body part for medical applications.

Despite these various advantages, OPDs are limited in their use due to their high dark current (higher by several orders of magnitude than that of a silicon-based photodiode, for example), which results in degrading the measured signal-to-noise ratio, or SNR. Now, the SNR must be significant to be able to detect weak signals such as those obtained in physiological parameter monitoring applications, and to obtain a reliable and robust measurement.

To improve the SNR of an OPD, the document “Vacuum-Processed Small Molecule Organic Photodetectors with Low Dark Current Density and Strong Response to Near-Infrared Wavelength” by C-C. Lee et al, Adv. Optical Mater. 2020, vol. 8, Issue 17, p. 2000519, provides the use of low-noise organic materials. The use of such materials is however constraining.

The document “Organic narrowband near-infrared photodetectors based on intermolecular charge-transfer absorption” by Siegmund, B. et al., Nat. Commun 8, 15421 (2017) provides improving the SNR of an OPD by forming, within it, an optical cavity to trap the light to be detected in the OPD stack and cause a plurality of round trips of light through the organic photosensitive material of the OPD, and thus increase the absorption of light by the photosensitive material to, ultimately, increase the level of electrical output signal obtained while keeping an identical noise level. This solution is effective for a given wavelength defined by the thickness of the optical cavity. However, when a plurality of wavelengths are to be detected by the OPD or when the wavelength to be detected is likely to change, the resonant cavity no longer offers any advantage.

Similar problems can also be encountered in other fields such as that of optical communications, for example when it comes to optimizing the coupling between an optical guide element, for example a waveguide, and an electronic component for converting light into an electrical signal.

### SUMMARY OF THE INVENTION

There is a need to provide a solution overcoming at least part of the disadvantages discussed hereabove.

An embodiment overcomes all or part of these disadvantages and provides a photodetector device comprising at least:
an optical guide element;
an electronic component for converting light into an electrical signal, configured to detect light at least on the side of a first surface of the electronic component arranged in front of the optical guide element and on the side of a second surface of the electronic component opposite to the first surface;
a reflective layer arranged on the side of the second surface of the electronic component;
an optical component arranged between the reflective layer and the optical guide element and at least partially transparent to the light to be detected by the electronic component;
and wherein the reflective layer forms at least one reflective surface conformal to a non-planar surface of the optical component having the reflective layer arranged thereon.

According to a specific embodiment, the electronic component comprises at least one photodiode.

According to a specific embodiment, the electronic component comprises at least one layer of organic material.

According to a specific embodiment, the optical component comprises at least one concave portion.

According to a specific embodiment, the reflective surface forms at least one spherical or conical or hyperbolic or parabolic mirror.

According to a specific embodiment, the reflective layer comprises at least one metal layer and/or at least one Bragg mirror.

According to a specific embodiment, the photodetector device further comprises at least one substrate at least partially transparent to the light to be detected by the electronic component and arranged between the optical guide element and the electronic component.

According to a specific embodiment, the optical guide element comprises at least one microneedle at least partially transparent to the light to be detected by the electronic component, or at least one waveguide.

According to a specific embodiment, the optical guide element comprises a base at least partially transparent to the light to be detected by the electronic component, and a plurality of microneedles at least partially transparent to the light to be detected by the electronic component and each comprising a first end integral with the base, the base being arranged between the electronic component and the microneedles.

According to a specific embodiment, the photodetector device comprises a plurality of distinct electronic components.

According to a specific embodiment, each of the electronic components is arranged vertically in line with one of the microneedles, or each of the electronic components is arranged vertically in line with a group of microneedles, or a group of electronic components is arranged vertically in line with each of the microneedles, or electronic components are arranged vertically in line with spaces between microneedles.

There is also provided a physiological parameter measurement device comprising at least one photodetector device such as previously described.

There is also provided a photovoltaic device comprising at least one photodetector device such as previously described.

There is also provided a method of manufacturing a photodetector device, comprising at least:
the forming of at least one optical guide element;
the forming of at least one electronic component for converting light into an electrical signal, configured to detect light at least on the side of a first surface arranged in front of the optical guide element and on the side of a second surface opposite to the first surface;
the forming of at least one reflective layer arranged on the side of the second surface of the electronic component;
the forming of at least one optical component arranged between the reflective layer and the optical guide element and at least partially transparent to the light to be detected by the electronic component;
and wherein the reflective layer is formed in such a way that it forms at least one reflective surface conformal to a non-planar surface of the optical component having the reflective layer arranged thereon.

According to a specific embodiment:
the electronic component and the reflective layer are formed on the optical guide element, or
the electronic component and the reflective layer are formed on a substrate at least partially transparent to the light to be detected by the electronic component, the substrate then being bonded to the optical guide element.

### BRIEF DESCRIPTION OF THE DRAWINGS

The foregoing features and advantages, as well as others, will be described in detail in the rest of the disclosure of specific embodiments given as an illustration and not limitation with reference to the accompanying drawings, in which:

FIG. 1 schematically shows an example of a photodetector device according to a specific embodiment;

FIG. 2 shows curves showing the light power received by an electronic photoconversion component configured to detect light on two opposite surfaces, as a function of the radius of the electronic photoconversion component, in a photodetector device according to a specific embodiment;

FIG. 3, FIG. 4, FIG. 5, FIG. 6, FIG. 7, FIG. 8, FIG. 9, and FIG. 10 show steps of an example of a method of manufacturing a photodetector device according to a specific embodiment.

### DESCRIPTION EMBODIMENTS

Like features have been designated by like references in the various figures. In particular, the structural and/or functional features that are common among the various embodiments may have the same references and may dispose identical structural, dimensional and material properties.

In the drawings, to make their reading easier, the different elements and the different layers of materials are not shown to the same scale with respect to one another.

For clarity, only those steps and elements which are useful to the understanding of the described embodiments have been shown and are described in detail.

Unless indicated otherwise, when reference is made to two elements connected together, this signifies a direct connection without any intermediate elements other than conductors, and when reference is made to two elements coupled together, this signifies that these two elements can be connected or they can be coupled via one or more other elements.

In the following description, where reference is made to absolute position qualifiers, such as the terms “front”, “back”, “top”, “bottom”, “left”, “right”, etc., or relative position qualifiers, such as the terms “top”, “bottom”, “upper”, “lower”, etc., or orientation qualifiers, such as “horizontal”, “vertical”, etc., reference is made unless otherwise specified to the orientation of the drawings. However, these terms do not presume the actual position and orientation of the device during its use.

Unless specified otherwise, the expressions “about”, “approximately”, “substantially”, and “in the order of” signify plus or minus 10%, preferably of plus or minus 5%. Further, unless otherwise specified, the given ranges of values include the limits of these ranges.

Throughout the document, the expression “at least partially transparent” is used to characterize the fact that an element can be crossed by at least part (for example, at least 40% or at least 50% or at least 70% or at least 90%) of the light received at the inlet of this element.

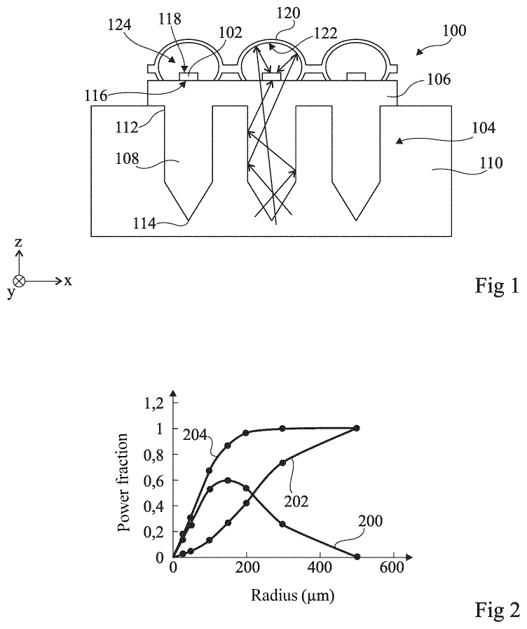

An example of a photodetector device 100 according to a specific embodiment is described hereafter in relation with FIG. 1.

In this example of embodiment, device 100 corresponds to a physiological parameter measurement device provided with a portion formed of microneedles intended to be inserted into tissue, through the outer surface of the skin.

Device 100 comprises at least one electronic component 102 for converting light into an electrical signal, that is, a photoconversion or photodetection electronic component. In the described embodiment, device 100 comprises a plurality of distinct electronic components 102 spaced apart from one another. However, device 100 may comprise a single electronic component 102.

In the described example of embodiment, each of electronic components 102 comprises at least one photodiode. More particularly, in the described example, each electronic component 102 corresponds to a photodiode. Further, in this example, electronic components 102 are organic in nature, that is, comprise one or more organic materials, and correspond to OPDs.

In the described example, each of electronic components 102 comprises first and second electrodes, one corresponding to the anode and the other corresponding to the cathode of the photodiode formed by electronic component 102, based on at least one electrically-conductive material. The electrodes are at least partially transparent (and preferably totally or almost totally transparent) to at least one type of light intended to be detected by electronic component 102. Further, each of electronic components 102 comprises at least one photodetection layer, or photosensitive layer, comprising at least one organic semiconductor material and arranged between the first and second electrodes. The thickness (dimension parallel to the Z axis in FIG. 1) of each electronic component 102 is, for example, in the range from 50 nm to 500 nm. As an example, the electrodes of each of electronic components 102 may comprise at least one of the following materials: Al, Ag, ITO, SnO₂, etc. The photodetection layer of each electronic component 102 may comprise, depending on the wavelength(s) to be detected, at least one of the following types of material: CuPc (copper phthalocyanine), ZnPc (zinc phthalocyanine), C₆₀, ClAlPc (chloroaluminum phthalocyanine), PCBM, PbPc (lead phthalocyanine), etc.

Device 100 further comprises at least one optical guide element 104. In the described example of embodiment, optical guide element 104 comprises a base 106 at least partially transparent (and preferably totally or almost totally transparent) to the light intended to be detected by electronic components 102, as well as a plurality of microneedles 108 also at least partially transparent (and preferably totally or almost totally transparent) to the light intended to be detected by electronic components 102. Microneedles 108 are here intended to be inserted into tissue 110 corresponding to surface layers of the skin. Each of microneedles 108 comprises a first end 112 integral with base 106 and a point-shaped second end 114.

More particularly, in the described example, each microneedle 108 comprises a cylindrical portion extending from the first end 112 and continuing in a tapered portion to the second end 114. As a variant, shapes of microneedles 108 different from those described hereabove are possible. For example, the cross-section of microneedles 108 may have a shape other than a disk. Further, shapes other than a point are conceivable, for example to orient optical guide element 104 or to couple it with a local diffuser arranged at the end of microneedles 108.

In the described example of embodiment, microneedles 108 are configured to guide the light scattered from tissue 110 to the surface of base 106 having electronic components 102 arranged thereon. The conical shape of the tips of microneedles 108 enables microneedles 108 to penetrate the skin well, and also to ensure a good collection of the guided light in the cylindrical section of microneedles 108. In the described example, the guiding of light within microneedles 108 is achieved due to the difference in refractive index between the material of microneedles 108 and tissue 110. As an example, the height of each of microneedles 108 (dimension parallel to the Z axis in FIG. 1) may be in the range from 100 μm to 3 mm. The cross-section of the cylindrical section of each of microneedles 108 has, for example, in a plane perpendicular to their height (plane parallel to the (X, Y) plane in the example of FIG. 1), a diameter in the range from 50 μm to 900 μm. The pitch of microneedles 108, that is, the distance separating the axes of revolution of two adjacent microneedles 108, may be in the range from approximately 100 μm to several millimeters, or greater than or equal to 500 μm. According to an embodiment, microneedles 108 may comprise a biocompatible material such as a polymer, for example polymethyl methacrylate or PMMA, or PLGA (poly (lactic-co-glycolic acid)).

When device 100 is intended for applications other than the measurement of physiological parameters by photodetection of light propagating through the skin, optical guide element 104 may comprise base, 106 to which are optically coupled one or more elements at least partially transparent (and preferably totally or almost totally transparent) to the light to be detected by electronic components 102, which element(s) may be different from microneedles 108.

Thus, optical guide element 104 may comprise, for example, at least one microneedle, or at least one waveguide (example of an application other than the measurement of physiological parameters) or other types of element depending on the envisaged application (for example the photovoltaic field), and optical guide element 104 may or not comprise base 106.

Each of electronic components 102 is configured to detect light at least on the side of a first surface 116 arranged in front of optical guide element 104 and also on the side of a second surface 118 opposite to first surface 116. This light detection on the side of each of surfaces 116, 118 is due, in this example of embodiment, to the fact that electronic components 102 correspond to OPDs having their organic photodetection layer(s) detecting light on the side of each of the electrodes (first electrode arranged on the side of first surface 116 and second electrode arranged on the side of second surface 118). As a variant, this light detection from both surfaces 116, 118 of electronic components 102 can be achieved by using other types of electronic components 102 configured to detect light from two opposite sides, or surfaces.

In the described example of embodiment, the first surface 116 of each of electronic components 102 is directly arranged against optical guide element 104, and more particularly against the base 106 of optical guide element 104. As a variant, it is possible for at least one element at least partially transparent, or preferably totally or almost totally transparent, to the light to be detected by electronic components 102, for example a substrate made of glass or of any other transparent or semi-transparent material, to be interposed between electronic components 102 and optical guide element 104, such as for example conical bases having the microneedles arranged thereon, and with a possible baseplate having the conical bases resting thereon.

Device 100 further comprises at least one reflective layer 120 arranged on the side of the second surfaces 118 of electronic components 102 and forming a reflective surface 122 arranged in front of electronic components 102. This surface 122 is said to be reflective due to the fact that it is configured to reflect at least part of, and preferably all or almost all, the light transmitted from optical guide element 104 and which has not been absorbed by the first surfaces 116 of electronic components 102, to increase the total amount of light absorbed by electronic components 102. Reflective layer 120 comprises, for example, at least one metal such as silver or aluminum. The thickness of reflective layer 120 is, for example, in the range from 50 nm to 500 nm.

As a variant, reflective layer 120 may comprise at least one Bragg mirror configured to reflect the wavelength(s) of interest intended to be detected by electronic components 102.

In any case, the properties of reflective layer 120 (material(s) used, thickness, shape, etc.) may be such that reflective surface 122 reflects as much light as possible in order to have the lowest possible light loss at this reflective layer 120.

Device 100 further comprises at least one optical component 124 arranged between reflective layer 120 and optical guide element 104, and more particularly between each of electronic components 102 and reflective layer 120. In the example of FIG. 1, device 100 comprises a plurality of optical components 124, each arranged vertically in line with one of microneedles 108 and also arranged between one of electronic components 102 and reflective layer 120. The pitch (distance between the centers of two adjacent optical components 124) with which optical components 124 are formed may be equal to that of microneedles 108. Optical components 124 are at least partially transparent, and preferably totally or almost totally transparent, to the light to be detected by electronic components 102. According to an example of embodiment, optical components 124 comprise a resin-type polymer (for example, PMMA or PLGA) or an oxide such as SiO₂ or SiN or any other suitable material. Further, the thickness of each of optical components 124 (that is, their dimension parallel to the Z axis in the example of FIG. 1) is, for example, in the range from 50 μm to 2 mm, or greater than or equal to 100 μm.

Reflective layer 120 is arranged on optical components 124 in such a way that reflective surface 122 is conformal to a non-planar surface of optical components 124 and thus achieves a light reflection according to a desired directivity and/or focus defined by the shape of the non-planar surface of optical components 124. Thus, the geometry of reflective surface 122 in front of each microneedle 108 depends on that of the non-planar surface of each optical component 124. In the described example of embodiment, each optical component 124 forms a concave surface having reflective layer 120 arranged thereon, this shape corresponding to that of reflective surface 122. For example, optical components 124 may be such that reflective surface 122 forms, in front of each microneedle 108, at least one spherical mirror, or spherical cap, which may also be conical, or hyperbolic, or advantageously parabolic. Other shapes are also possible: cube corner, ellipse, etc. As a variant, each optical component 124 may have another non-planar shape adapted for reflective surface 122 to perform a desired light reflection towards electronic components 102. For example, optical components 124 may be such that, combined with reflective surface 122, they enable to locally increase the directivity of light, to reflect it with a suitable angle towards electronic components 102.

For example, when device 100 is used in the photovoltaic field, having a reflective surface 122 forming a parabolic mirror enables to cover a much wider spectral range than when microlenses are used (limited by the reflective properties of the materials used). These advantages are obtained for all wavelengths.

Thus, optical components 124 combined with the reflective surface 122 may be configured to focus light onto the side of the second surface 118 of each of electronic components 102.

In device 100, in order to limit crosstalk between electronic components 102, it is possible to decrease as much as possible the thickness of base 106, and more generally to decrease the distance between electronic components 102 and the first ends 112 of microneedles 108, to prevent for light originating from one of microneedles 108 to be detected by an electronic component 102 different from that arranged vertically in line with this microneedle.

As an variant of the above-described embodiment, optical guide element 104 may correspond to a waveguide. The device 100 according to such a variant may, for example, be used in the field of optical communications in order to optimize the optical coupling between electronic components 102 and the waveguide corresponding to optical guide element 104.

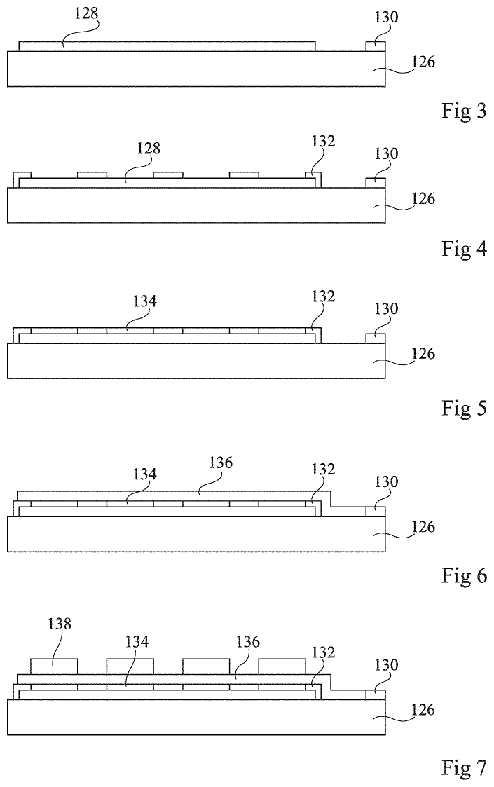

Curves shown in FIG. 2 show the fraction of light power originating from one of microneedles 108 and received by one of electronic components 102 arranged vertically in line with this microneedle 108, as a function of the radius (in microns) of electronic component 102 (here assuming that electronic component 102 has, in a plane perpendicular to the axis of revolution of microneedle 108, a disk-shaped cross-section). In FIG. 2, the received power fraction is defined as being the ratio between the detected light power and the total light power initially emitted from microneedle 108. Curve 200 represents the light power fraction received through the second surface 118 of electronic component 102, curve 202 represents the light power fraction received through the first surface 116 of electronic component 102, and curve 204 represents the sum of the light power fractions received through the two surfaces 116, 118 of electronic component 102. These values are obtained for:
a microneedle 108 having a diameter equal to 400 μm from which a luminous flux is emitted with a 25° half-angle of aperture;
a reflective surface 122 having a parabolic conical shape with a radius equal to 1 mm, a height (dimension parallel to the axis of revolution of microneedle 108) equal to 0.312 mm, and a focal length equal to 0.2 mm, electronic component 102 being centered on the focal point of reflective surface 122.

In this case, when the radius of electronic component 102 is equal to 500 μm, 100% of the light is detected by the first surface 116 of electronic component 102. By decreasing the diameter of electronic component 102, 100% of the light is still detected by electronic component 102 when the radius of electronic component 102 is in the range from 500 μm to 220 μm, due to the reflection of light on reflective surface 122 and its deflection towards the second surface 118 of electronic component 102. These curves show that it is possible to greatly decrease the dimensions of electronic component 102, and thus, in the case of an electronic component 102 corresponding to an OPD, to greatly decrease its dark current and thus greatly increase its SNR, while keeping a high light detection rate.

As a variant, it is possible for one or more electronic components 102 to be arranged, rather than opposite microneedle(s) 108, alongside or between them. Thus, it is for example possible to dissociate the information coming from the surface of tissue 110 from that coming from inside tissue 110, and thus to measure the potential information in the space between microneedles. In such a variant, electronic component(s) 102 may be arranged on base 106. Further, in such a variant, electronic component(s) 102 may correspond to one or more structured OPDs.

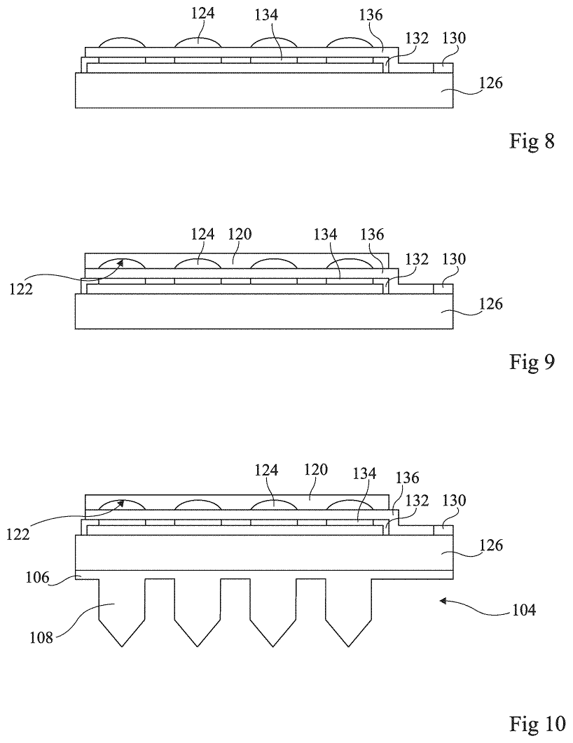

An example a method of manufacturing device 100 is described hereafter in relation with FIGS. 3 to 10.

In this example, electronic components 102, reflective layer 120, and optical components 124 are formed on a substrate 126 at least partially transparent, and preferably totally or almost totally transparent, to the light intended to be detected by electronic components 102. The thickness of substrate 126 is, for example, equal to a few hundred microns. For example, substrate 126 may comprise glass.

As a variant, it is possible for electronic components 102, reflective layer 120, and optical components 124 to be formed directly on optical guide element 104, as is the case in the example of FIG. 1.

In the described example of embodiment, electronic components 102 correspond to OPDs. Thus, in this example, a first transparent or semi-transparent electrode 128, that is, an electrode capable of letting through at least part of the light intended to be detected by electronic components 102, is formed on substrate 126. First electrode 128 corresponds, for example, to the anode of electronic components 102. At least one contact pad 130 having a second electrode of electronic components 102 intended to be electrically coupled thereto is also formed on substrate 126, next to first electrode 128 (see FIG. 3).

In the described example, first electrode 128 is common to the various electronic components 102. As a variant, it is possible for each electronic component 102 to comprise a first electrode 128 distinct and electrically insulated from the first electrodes 128 of the other electronic components 102, or for a plurality of distinct first electrodes 128 electrically insulated from one another to be formed on substrate 126, each of them being coupled to a plurality of electronic components 102.

Insulating portions 132, comprising, for example, resin, are then formed, for example by deposition, at the periphery of first electrode 128 and between locations of the future active photodetection regions of electronic components 102, that is, between the locations where the portions of the layer(s) intended to ensure light detection will be arranged (see FIG. 4). The insulating portions 132 arranged on the edges of first electrode 128 are intended to electrically insulate first electrode 128 from the electrical connection that will be formed between the second electrode of electronic components 102 and contact pad 130.

One or more light detection layers 134, here comprising at least one organic material, are then deposited on first electrode 128, between insulating portions 132 (see FIG. 5). This deposition is, for example, implemented through a stencil to localize the deposition of this or these light detection layers 134 at the desired locations, between insulating portions 132.

A second transparent or semi-transparent electrode 136 is then formed on light detection layer(s) 134 and insulating portions 132. This second electrode 136 corresponds, for example, to the cathode of electronic components 102. A portion of this second electrode 136 is deposited on at least one of insulating portions 132 arranged on one of the edges of first electrode 128 and on a portion of substrate 126 so as to be in contact with contact pad 130 (see FIG. 6).

In the described example, second electrode 136 is common to the various electronic components 102. As a variant, it is possible for each electronic component 102 to comprise a second electrode 136 distinct and electrically insulated from the second electrodes 136 of the other electronic components 102, or for a plurality of distinct second electrodes 136 electrically insulated from one another to be formed, each of them being coupled to a plurality of electronic components 102.

At this stage of the method, the forming of electronic components 102 is complete.

Although not shown, at least one transparent or semi-transparent encapsulation layer may then be deposited on electronic components 102.

Optical components 124 are then formed on electronic components 102. In the described example of embodiment, pads 138 of the material(s) intended to form optical components 124, for example transparent or semi-transparent resin pads, are for example formed by deposition over second electrode 136 (see FIG. 7). In the presence of an encapsulation layer covering electronic components 102, pads 138 are formed on this encapsulation layer.

A creep step can then be implemented to give pads 138 the desired shape and thus form optical components 124 (see FIG. 8).

Reflective layer 120 is then formed, for example by deposition, on optical components 124 and on the portions of second electrode 136 not covered by optical components 124 (see FIG. 9).

Device 100 is completed by transferring the structure created onto guide element 104 comprising, in the described example of embodiment, base 106 and microneedles 108. This transfer corresponds, in the described example, to a bonding of substrate 126 to base 106 (see FIG. 10).

In a variant, electronic components 102 may be formed in such a way that they are configured to detect light of different wavelengths. In this case, the deposited light detection layer(s) 134 are, for example, different according to the wavelength(s) intended to be detected.

In the above-described examples of embodiments, device 100 comprises a plurality of electronic components 102, each arranged vertically in line with one of microneedles 108. As a variant, device 100 may comprise a single electronic component 102, corresponding, for example, to a single photodiode, arranged vertically in line with the set of microneedles 108. According to another variant, device 100 may comprise a plurality of electronic components 102 such that each of them is arranged vertically in line with a group of microneedles 108. According to another variant, device 100 may comprise a plurality of electronic components 102 such that a group of electronic components 102 is arranged vertically in line with each of microneedles 108.

In the previously-described example of embodiment, device 100 corresponds to a device used in the field of healthcare to monitor, or measure, physiological and/or therapeutic parameters. In device 100, light is a measurement modality used to determine and monitor physiological parameters of interest, such as heart rate, blood oxygen levels, blood glucose, SpO2 or SaO2. The light guided in optical guide element 104 and detected by electronic components 102 corresponds to light propagating in tissue 110 and which originates, for example, from a light source external to tissue 110. The light guided in optical guide element 104 has, prior to its entering optical guide element 104, interacted with the tissue of interest, which carries the information enabling to measure the targeted physiological parameters.

Whatever the targeted application, device 100 enables to maximizes photon collection on electronic component(s) 102 while keeping, when electronic components 102 correspond to photodiodes, a decreased detection surface area to minimize the dark current and thus increase the SNR of the photodiodes so as to make the measurement more reliable and robust, and this notwithstanding the low strength of the collected optical signal due to the high absorption rate in the skin and to the scattering of light in tissue 110.

As an example, as compared with a photodetector device which would not be equipped with reflective surface 122 enabling light to be reflected towards the second surface 118 of electronic components 102, it is possible, with device 100, to decrease the active photodetection surface area of electronic components 102 for example by a factor 5.2, which decreases the dark current by the same amount, without decreasing the fraction of light power detected by electronic components 102.

These advantages are also obtained whatever the length of the detected light, and even when the light is polychromatic. Further, these advantages are obtained without any specific constraints on the organic materials likely to be used for the forming of electronic components 102, and device 100 is compatible with materials optimized for the desired photodetection.

Device 100 enables to optimize the detection of the light guided by optical guide element 104 due to the judicious use of non-planar reflective surface 122, which, combined with one or more photodetector electronic components 102, detecting light both on the side of optical guide element 104 and on the side of reflective surface 122, enables to increase the amount of light sent to electronic component(s) 102, since the light reaching reflective surface 122 is recovered and reflected towards electronic component(s) 102 due to the light reflection and focusing properties of reflective surface 122.

In this example of device 100, the light originating from tissue 110 is guided by microneedles 108. Part of this light may be absorbed by the first surface 116 of electronic components 102, while that which is not absorbed is reflected by reflective surface 122 and sent back to the second surface 118 of components 102 for detection.

The fact of having electronic components 102 corresponding to photodiodes enables to benefit from their integrability properties. For example, OPDs have the advantage of being robust to the steps implemented for the forming of optical components 124.

Device 100 can be used for fields of applications other than those previously described, for example within “smart pixels” in displays to optimize the SNR of these pixels, or in any field requiring the forming of a photodiode or of a photodetector, and particularly when the signal received by the photodiode is weak, or also in the photovoltaic field.

Various embodiments and variants have been described. Those skilled in the art will understand that certain features of these various embodiments and variants may be combined, and other variants will occur to those skilled in the art.

Finally, the practical implementation of the described embodiments and variants is within the abilities of those skilled in the art based on the functional indications given hereabove. For example, the precise nature of the implemented deposition and etch steps can be selected according, in particular, to the material(s) to be deposited or to be etched, as well as to the thicknesses of the materials to be deposited or to be etched.

1. Photodetector device comprising at least:
an optical guide element;
an electronic component for converting light into an electrical signal, configured to detect light at least on the side of a first surface of the electronic component arranged in front of the optical guide element and on the side of a second surface of the electronic component opposite to the first surface;
a reflective layer arranged on the side of the second surface of the electronic component;
an optical component arranged between the reflective layer and the optical guide element and at least partially transparent to the light to be detected by the electronic component;
and wherein the reflective layer forms at least one reflective surface conformal to a non-planar surface of the optical component having the reflective layer arranged thereon.

2. Photodetector device according to claim 1, wherein the electronic component comprises at least one photodiode.

3. Photodetector device according to claim 1, wherein the electronic component comprises at least one layer of organic material.

4. Photodetector device according to claim 1, wherein the optical component comprises at least one concave portion.

5. Photodetector device according to claim 1, wherein the reflective surface forms at least one spherical or conical or hyperbolic or parabolic mirror.

6. Photodetector device according to claim 1, wherein the reflective layer comprises at least one metal layer and/or at least one Bragg mirror.

7. Photodetector device according to claim 1, further comprising at least one substrate at least partially transparent to the light intended to be detected by the electronic component and arranged between the optical guide element and the electronic component.

8. Photodetector device according to claim 1, wherein the optical guide element comprises at least one microneedle at least partially transparent to the light to be detected by the electronic component, or at least one waveguide.

9. Photodetector device according to claim 8, wherein the optical guide element comprises a base at least partially transparent to the light to be detected by the electronic component and a plurality of microneedles at least partially transparent to the light to be detected by the electronic component and each comprising a first end integral with the base, the base being arranged between the electronic component and the microneedles.

10. Photodetector device according to claim 1, comprising a plurality of distinct electronic components.

11. Photodetector device according to claim 9, comprising a plurality of distinct electronic components, and wherein each of the electronic components is arranged vertically in line with one of the microneedles, or wherein each of the electronic components is arranged vertically in line with a group of microneedles, or wherein a group of electronic components is arranged vertically in line with each of the microneedles, or wherein electronic components are arranged vertically in line with spaces between microneedles.

12. Physiological parameter measuring device comprising at least one photodetector device according to claim 1.

13. Photovoltaic device comprising at least one photodetector device according to claim 1.

14. Method of manufacturing a photodetector device, comprising at least:
the forming of at least one optical guide element;
the forming of at least one electronic component for converting light into an electrical signal, configured to detect light at least on the side of a first surface arranged in front of the optical guide element and on the side of a second surface opposite to the first surface;
the forming of at least one reflective layer arranged on the side of the second surface of the electronic component;
the forming of at least one optical component arranged between the reflective layer and the optical guide element and at least partially transparent to the light to be detected by the electronic component;
and wherein the reflective layer is formed in such a way that it forms at least one reflective surface conformal to a non-planar surface of the optical component having the reflective layer arranged thereon.

15. Manufacturing method according to claim 14, wherein:
the electronic component and the reflective layer are formed on the optical guide element, or
the electronic component and the reflective layer are formed on a substrate at least partially transparent to the light to be detected by the electronic component, the substrate then being bonded to the optical guide element.
